(19) United States
(12) Patent Application Publication
(10) Pub. No.: US 2021/0236422 A1
(43) Pub. Date: Aug. 5, 2021

(54) NASAL SPRAY FORMULATION HAVING EPISTAXIS PREVENTION PROPERTIES

(71) Applicant: David HAMPSON, Milton, GA (US)

(72) Inventor: David HAMPSON, Milton, GA (US)

(21) Appl. No.: 17/163,477

(22) Filed: Jan. 31, 2021

Related U.S. Application Data
(60) Provisional application No. 62/967,981, filed on Jan. 30, 2020.

(51) Int. Cl. A61K 9/00; A61K 47/44; A61K 36/28; A61K 36/23

(52) U.S. Cl. CPC A61K 9/0073; A61K 9/0043; A61K 36/23; A61K 36/28; A61K 47/44

(57) ABSTRACT

A therapeutic nasal moisturizing formulation for moisturizing the epidermal surface of the nasal cavity and for the prevention of epistaxis. The formulation includes Medium Chain Triglyceride (MCT) coconut oil and tinctures of Achillea millefolium and Pimpinella saxifraga in an approximately 8:1:1 ratio. The MCT oil is preferably fractionated and/or refined to include only caprylic acid, capric acid, and lauric acid. Tinctures can be diluted three times into 1% potency and alcohol may be removed from the tinctures. The formulation may be combined in an approximately 8 to 1 to 1 ratio. A method for the preparation of the formulation and a method of nasal moisturization for the prevention of epistaxis. The method steps include providing the therapeutic formulation and spraying the formulation into the nasal cavity into the nostril(s) of a patient, thereby moisturizing the cavity through the long-lasting moisturization of nasal epidermis and prevention of drying and cracking of nasal epidermis.

### CROSS-REFERENCE TO RELATED APPLICATIONS

To the full extent permitted by law, the present United States Non-provisional patent application hereby claims priority to and the full benefit of, U.S. Provisional Application No. 62/967,981 filed on Jan. 30, 2020, entitled “NASAL SPRAY FORMULATION HAVING EPISTAXIS PREVENTION PROPERTIES “COCONOSE”, which is incorporated herein by reference in its entirety.

### BACKGROUND OF THE DISCLOSURE

### Technical Field of the Disclosure

The instant disclosure generally relates to spray and aerosol drugs for use in the nasal cavity. More particularly, to a nasal spray for the prevention of epistaxis, the nasal spray containing oil derived from fruit of Palmaceae, namely coconut oil.

### Description of the Related Art

Epistaxis is defined as acute hemorrhage from the nostril, nasal cavity, or nasopharynx—a nosebleed. Simply put, epistaxis is the loss of blood from the tissue that lines the inside of a nose. Epistaxis events are common. Some 60% of people will have at least one nosebleed in their lifetime. The location of the nose in the middle of the face and the large number of blood vessels close to the surface in the lining of your nose make it an easy target for injury and epistaxis. Although seeing blood coming out of a person's noise can be alarming, most epistaxis events are not serious and can be managed at home. Epistaxis events may begin anywhere in the nasal cavity, but some may begin or persist in the back of the nose. An anterior epistaxis begins in the front of the nose on the lower part of the wall that separates the two sides of the nose (septum). Capillaries and small blood vessels in this area are fragile and can easily break. An anterior epistaxis is the most common form, though usually less serious. Anterior epistaxis is most common in children. Posterior epistaxis occurs deep inside the nose and can result in heavy bleeding from the nose and even bleeding which can flow down the back of the throat. Posterior epistaxis usually involves large blood vessels and results in heavy bleeding which can be dangerous. Some posterior epistaxis events may even need medical attention, especially if the bleeding occurs after an injury and the bleeding has not stopped after 20 minutes of applying direct pressure to the nose. While normally treated through a variety of everyday measures normally used to contain and stop bleeding in other areas of a body, it is a frequent emergency department complaint and often causes significant anxiety in patients and clinicians. However, the vast majority of patients who present to an emergency department with epistaxis (likely more than 90%) may be successfully treated by an emergency physician.

Given the consistent volume of air constantly passing through such a small surface area of epidermis within the nasal cavity, human and environmental factors can increase the frequency and/or duration of epistaxis. Factors such as dry air, cold air, viral/bacterial infection, allergic reaction to environmental contaminants, the like, and combinations thereof may increase the likelihood of or actually cause an epistaxis event, even absent an injury to the nose. Alone or in combination with these factors, nose picking, colds and sinusitis, blowing the nose forcefully, inserting foreign objects into the nose, injury to the nose/face, allergic inflammation, blood thinning drugs, cocaine and other drugs inhaled through the nose, chemical irritants, high altitudes, deviated septum, and even frequent use of nasal medications and sprays may also cause epistaxis. Less common, though sometimes more serious, causes of epistaxis include but are not limited to alcohol use, bleeding disorders, high blood pressure, atherosclerosis, facial/nasal surgery, nasal tumors/polyps, immune thrombocytopenia, leukemia, hereditary hemorrhagic telangiectasia, and pregnancy.

The large number of possible causes of epistaxis may cause difficulty in diagnosis if a patient suffers frequent and/or persistent epistaxis. Patients reporting frequent or persistent epistaxis are usually evaluated for diagnoses of one or more of the above likely causes, but may be discharged without a certain diagnosis or without a treatment plan offering relief from what may be a simple, yet difficult to treat cause: dry epidermis within the nasal cavity.

Common over-the-counter and prescription-based preparations for use as nasal moisturizers may include any number of saline, glycerin, alcohol, pharmaceuticals, natural or artificially added scents, and oils. These preparations may be applied to a cotton swab (e.g. Q-TIPS® cotton swabs) and penetrated into the nasal cavity, suspended into a dropper and dropped into a nasal cavity with the patient in a reclined position, or sprayed into the nasal cavity via a pump and/or aerosolizing nasal sprayer. These preparations may also include an over-the-counter medication alone or in combination with the above delivery liquids and gels. One medication for the treatment of epistaxis is oxymetazoline, sold under the brands AFRIN®, DRISTAN®, NEO-SYNEPHRINE®, VICKS SINEX®, and others. Each of these over-the-counter medications that include oxymetazoline include a warning against use over a long period of time and therefore may not be suitable for someone suffering recurrent epistaxis episodes because they may in fact increase the chances of epistaxis if used over a long period of time. Tranexamic acid is an orally taken medication that slows down or stops blood clots from being naturally broken down by the body, and may be another treatment for recurrent epistaxis, though use may have very serious side effects. Additional treatments intended to moisturize may include room humidifiers, nasal packing with gauze or inflatable latex balloons, cauterization via chemical or heat treatment, abstinence from blood-thinning medication (e.g. daily low-dose aspirin), foreign body removal (if present), surgical repair of broken nose or deviated septum, ligation, and nasal irrigators. While these treatments may offer some relief to patients suffering persistent or frequent epistaxis through temporarily introducing moisture into the nasal cavity, removing the cause of epistaxis, few actually offer prolonged benefit beyond a brief period such as a few hours. Furthermore, some of these may have serious immediate or side effects. Some topical preparations and moisturizing techniques may require frequent application of the preparation over the course of the day or use, respectively, and on the whole may even dry-out, defeat or minimize the cavity's self-moisturizing capabilities, or even further irritate the nasal cavity, thus exacerbating the possible cause of epistaxis and causing even more frequent or drastic events of epistaxis.

Properly moisturized skin is known to be less susceptible to breaking, cracking, and bleeding. Oils, either naturally produced by the human epidermis organ system or applied directly as a moisturizer to the human epidermis, may act as a sealant, as it may help to trap water into the skin to keep it moist. Oils have been investigated and used for centuries as a moisturizer for human epidermis. Historians and archaeologists have reported use of emu oil as a moisturizer by aboriginal tribes as early as 40,000 years ago, use of plant-based oils on skin by ancient Egyptians, and use of olive oil on skin by ancient Olympic athletes. While oils may be used as moisturizers in-and-of-themselves, they may also provide increased sealant properties if used atop or along with another moisturizing compound. A variety of oils, including coconut oils, have been investigated for use in moisturizing the nasal cavity and also for use for what may be antiviral, antibiotic, and even neurological benefits. However, an oil-based treatment for epistaxis is not known to exist, either in the marketplace or in the medical literature.

Many natural products, including plants, may have known benefits to human health and may possess long histories of use as homeopathic remedies. Some homeopathic remedies may generally be prepared by grinding, crushing, shredding, masticating, or otherwise processing plants or other natural products, then preparing tinctures using a mixture of the processed natural product and alcohol, vinegar, glycerin, the like and/or combinations thereof. Other methods to prepare such remedies may include powder form (capsules) or teas. For a large portion of natural products, there may be known protocol for the creation of tinctures so as to maintain a common reference point among a variety of manufacturers of homeopathic remedies and/or tinctures. This may include certain tincture bases (e.g. 70% ethyl alcohol and 30% water), temperatures, tincturing times, assay results, chemical analysis, concentration, pH, the like and/or combinations thereof. In the United States, these protocols and standards are assembled into a homeopathic authoritative encyclopedia known as Homeopathic Pharmacopoeia of the United States (HPUS). Most helpful to epistaxis may be the provision of remedies known to possess wound-healing and/or moisturizing properties.

Achillea millefolium, commonly called common yarrow, is a rhizomatous, spreading, upright to mat-forming perennial that is considered by many to be an aggressive weed. A perennial herb, having a slender, creeping brown rhizome, with numerous filiform rootlets of more or less reddish color, and long. reddish stolons, with a succulent scale at each node, The stem, 0.3-1.2 m high, is erect, stiff, slightly striate, branched above, more or less covered with white, shaggy hair. The lower leaves are lanceolate in outline, up to 350 mm long and up to 40 mm wide, usually 3-pinnate with 15 or more leaflets on either side of the only slightly rounded rachis, The petiole is about 25 rnrn long; the petiole and rachis are grooved on the upper surface, Leaves are sparsely to relatively densely hairy, Leaves on the middle and upper part of the stem are 10 to 20 mm wide, alternate, sessile, with slightly enlarged basil leaflets that are somewhat ear-shaped. The flowers, appearing from June to October, are 2-10 mm large and are gathered at the end of the stalk in compound tight corymbs. Scarce ligulate, peripheral, white or rose flowers. Yellow central tubular flowers. Involucre oblong, imbricate, pale-green. Individual flower heads are about 10 mm wide, with a cup shaped, 4 to 5 mm long involucre of usually three rows of bracts, which are finely fringed at the tip, and a membranous, whitish, brownish or blackish margin. The four or five ray florets are short, white, sometimes rose-colored; the eight to twelve disk flowers are bisexual and are individually subtended by bracts with dentate tips that are only slightly narrower than the bracts of the involucre. The outer ones are almost carinate, with a green midline and scarious margin, the inner ones are smaller and entirely membranous. The pistil is forked at the tip, with the hairy, truncate branches arching downwards, the elongated inferior ovary has a narrow dentate band of thickening in the upper margin. The disc florets consist of a yellowish-white, five-pointed corolla about 4 mm long and tubular at the base, with five stamens; the anthers are syngenesious. The pistil that emerges through them and the ovary are of similar appearance to the corresponding parts of the ray florets. The aerial parts have a bitter taste and develop an intensely aromatic odor when crushed.

A tincture may be made from the whole plant of Achillea millefolium according to The Homeopathic Pharmacopoeia of the United States (HPUS) under the monograph HPUS 6102, its latest revision being Nov. 2, 2013. The monograph for Achillea millefolium HPUS 6102, including the instructions for preparation of a 1× alcohol-based tincture thereof are hereby incorporated by reference. It has long understood properties for treatment of wounds, owing its Latin name to Achilles, who was said to have used yarrow for the wounds of his friends on the battlefield. In addition to treatment of wounds, references may be found in various versions of the Materia Medica for the prevention of nosebleeds, or epistaxis.

Pimpinella saxifraga, known as burnet-saxifrage, solid stem burnet saxifrage, or lesser burnet is a plant species in the family Apiaceae, a native of the British Isles and temperate Europe and Western Asia. It is neither a burnet, which its leaves resemble, nor a saxifrage although it has a similar herbal effect as a diuretic. A deciduous, perennial herb, with long, cylindrical, sub-fusiform, slightly fibrous, tough, woody root. grayish-yellow externally, white within, highly aromatic and pungent. The stem, 3-6 dm. high. is solid, round, striated, slightly downy, branched, furrowed and smooth. The leaves are petiolate, oblong, pinnatifid, finely dentate and smooth; the radical are roundish, the cauline linear. The white flowers appear in autumn.

A tincture may be made from the root of Pimpinella saxifrage according to HPUS under the monograph HPUS 7065. The monograph for Pimpinella saxifrage HPUS 7065, including the instructions contained therein for the preparation of a 1× alcohol-based tincture thereof are hereby incorporated by reference. It is an alcohol-based tincture and references may also be found in various versions of the Materia Medica for the prevention of epistaxis and reduction in nasal dryness.

However, as useful as each of these naturally derived tinctures may be for the treatment and prevention of persistent epistaxis, given that they each predominate as alcohol-based tinctures, the tinctures themselves may cause additional drying, cracking, or lack of moisture in the nasal cavity, as described above. So, while their ancient applications, as detailed in the Materia Medica and various other ancient homeopathic and medicinal resources, for wound healing and the prevention of epistaxis may indeed persist in their effectiveness into the modern times, the alcohol-based preparations may diminish, defeat, eliminate, or even exacerbate the symptom they are intended to treat, namely, epistaxis.

Thus, there is clearly a need to provide a nasal moisturizer that is capable of retaining moisture within the nasal cavity throughout the day without drying out or irritating the nasal cavity in the process, such as an alcohol-free oil-based preparation and/or formulation designed for such use to be used regularly, but minimally for the treatment of frequent or recurrent epistaxis.

The instant disclosure is designed to address at least some of the aspects of the problems discussed above. The nasal spray formulation having epistaxis prevention properties described herein may be designed to replace other varieties of nasal moisturizers, and may also be designed to use alongside existing methods and preparations/formulations of treatment. The disclosed nasal spray formulation having epistaxis prevention properties may be applied through a variety of application methods, including those mentioned above and those described below.

### SUMMARY

Briefly described, in a possibly preferred embodiment, the present disclosure overcomes the above-mentioned disadvantages and meets the recognized need for such a nasal moisturizer by providing a nasal moisturizing formula comprising naturally derived oils and liquids. Being naturally derived allows such a formulation to be made and marketed as a certified organic and/or homeopathic treatment which offers many advantages to both manufacturers and consumers/patients. A majority, by weight and/or volume, of the disclosed nasal spray formulation having epistaxis prevention properties comprises oils derived from a fruit of a Palmaceae, namely coconut oil. Other ingredients may include plant-derived tinctures having both medicinal and scent properties and dissolved in ethyl alcohol and water, vinegar, glycerol, diethyl ether, and/or propylene glycol. The fractional weight of triglycerides in the primary ingredient of the disclosed nasal spray formulation having epistaxis prevention properties may be selected for to include those having certain properties that one skilled in the art may know to be beneficial for a nasal spray formulation. For the lower-concentration ingredients, the plant chosen, the tincturing solution chosen, and the concentration of each may be selected by those skilled in the art of nasal preparation for their various properties in either scent or epistaxis prevention properties. Additionally, the concentration of lower-concentration ingredients may be increased or decreased via dilution and the alcohol contained within a tincture may be boiled off and/or evaporated prior to, during, or after mixing into the formulation of the disclosure. This may decrease the drying potential of the formulation so as to increase the potential of the formulation of the disclosure to prevent epistaxis, using the method of the disclosure.

In one aspect, the predominant ingredient, by volume and weight, of the disclosed formulation for epistaxis prevention may include oils derived from a fruit of a Palmaceae, namely coconut oil. The fractional weight of triglycerides in the primary ingredient of the disclosed nasal spray formulation having epistaxis prevention properties may be selected for to include those having certain properties that one skilled in the art may know to be beneficial for a nasal spray formulation. In a potentially preferred embodiment of the disclosed formulation, the formulation may include at 80% by volume a refined coconut oil mixture, fractionated and/or refined to include substantially only caprylic acid (C8:0), capric acid (C10:0), and lauric acid (C12:0) triglycerides. This formulation may be one of any number of combinations of fractionated and/or refined coconut or palm oils known, produced, manufactured, marketed, sold, and purchased as medium-chain triglyceride oil or just “MCT oil.” MCT oil may be purchased for any number of uses, including use as a dietary supplement. It is particularly useful and of note that it generally remains in liquid form at room temperature, something that may increase its utility as a delivery mechanism in a nasal moisturizer.

Another feature of the disclosed formulation for epistaxis prevention, may be that in select embodiments one of the additional, lower volume ingredients may offer the additional benefit of being naturally derived from plants. Natural derivation from plants may offer any number of benefits, including but not limited to: labeling as “organic”, “all natural”, the like, and combinations thereof; ease or ability to achieve regulatory approval; confidence in safety of a formulation having plant derived properties from consumers; actual safety; the like and combinations thereof.

In select embodiments of the formulation for epistaxis prevention described herein, the moisturizing formulation may be delivered through a variety of delivery mechanisms. These may include but are not limited to applying the formulation to a cotton swab and penetrating the cotton swab into the nasal cavity, suspending the formulation into a dropper and dropping into a nasal cavity with the patient in a reclined position, or spraying into the nasal cavity via a pump and/or aerosolizing nasal sprayer. Given the ease and convenience, a potentially preferred method of delivery of the disclosed formulation may be spraying into the nasal cavity via a pump and/or aerosolizing nasal sprayer.

These and other features of the disclosed formulation for epistaxis prevention will become more apparent to one skilled in the art from the prior Summary, and following Brief Description of the Drawings, Detailed Description, and Claims when read in light of the accompanying Detailed Drawings.

### BRIEF DESCRIPTION OF THE DRAWINGS

The present formulation, delivery apparatuses and methods of treatment will be better understood by reading the Detailed Description with reference to the accompanying drawings, which are not necessarily drawn to scale, and in which like reference numerals denote similar structure and refer to like elements throughout, and in which:

FIG. 1 is a cross-sectional drawing of a human nasal cavity with anatomical illustrations of major blood vessels which supply blood to the nasal cavity;

FIG. 2 is a perspective view of a patient applying a nasal spray to a nostril via a nasal spray device;

FIG. 3 is a flow chart of a method of preparation of a formulation for the prevention of epistaxis; and

FIG. 4 is a flow chart of a method of treatment for the prevention of epistaxis.

It is to be noted that the drawings presented are intended solely for the purpose of illustration and that they are, therefore, neither desired nor intended to limit the disclosure to any or all of the exact details of construction shown, except insofar as they may be deemed essential to the claimed disclosure.

### DETAILED DESCRIPTION

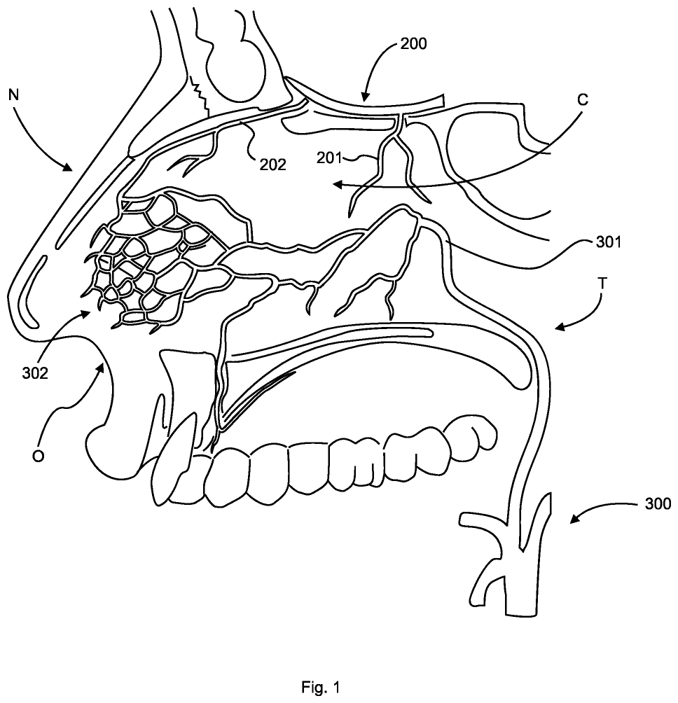

In describing the exemplary embodiments of the present disclosure, as illustrated in FIGS. 1-4, specific terminology is employed for the sake of clarity. The present disclosure, however, is not intended to be limited to the specific terminology so selected, and it is to be understood that each specific element includes all technical equivalents that operate in a similar manner to accomplish similar functions. Embodiments of the claims may, however, be embodied in many different forms and should not be construed to be limited to the embodiments set forth herein. The examples set forth herein are non-limiting examples, and are merely examples among other possible examples.

Referring now to FIGS. 1-4 by way of example and not limitation, various features of the human body are specifically illustrated or named and are not intended to be claimed as a feature of the disclosed method and formulation as herein described. These aspects of human form and anatomy are illustrated and described herein merely to adequately describe to one skilled in the art the purpose and methods of use of the disclosed method and formulation. Various anatomical features of the human anatomy may be specifically described herein by a formal, medical name, or by the more commonly understood name. By way of example a nose, nasal cavity, artery, vein, nostril, and the like may be described herein under their common or medical name, each of which are intended to be used as the meaning understood by those skilled in the art. Furthermore, conditions described herein are done so for exemplary purposes only and are not intended to be claimed as part of the disclosed method and formulation herein. These may also be described in various common or formal medical forms. By way of example and not limitation, these may include nosebleeds versus epistaxis. Finally, scientific and common names of plants, fruits, oils, the like and combinations thereof may be used interchangeably herein. By way of example and not limitation, medium chain triglyceride coconut oil may be referred to in various forms including but not limited to MCT Oil, MCT, MCT coconut oil, the like and/or combinations thereof.

Referring now to FIG. 1 specifically, therein is illustrated a cross-sectional view of a human nasal cavity of nose N and the various major blood vessels, arteries and veins, contained therein. Travelling from nostril O of nose N posteriorly and/or dorsally through nasal cavity C toward throat T, therein drawn is abundant external and internal carotid supply to the nasal cavity. Just inside nostril O along the septum of nose N is Kiesselbach's area 102 (also known as Little's area, Kiesselbach's plexus, or Kiesselbach's triangle) of the anterior septum. Kiesselbach's area 102 is a vascular network of the five arteries that supply the nasal septum which provides superficial anastomoses. External carotid branches supply the nose externally (via the facial artery shown therein Kiesselbach's area 102), and internal maxillary artery 100, including sphenopalatine artery 101, does so internally. Internal carotid branches are supplied via ophthalmic artery 200 to anterior ethmoidal artery 202 and posterior ethmoidal artery 201. Venous drainage occurs via facial veins as well as ophthalmic veins, which have valveless intracranial connections to the cavernous sinus and therefore relate to intracranial hematogenous spread of infection. FIG. 1 is supplied to provide an understanding of the blood supply to the nasal cavity and its corresponding relationship to the cause(s) of epistaxis, but is not described and/or disclosed as a feature of the disclosed formulation for prevention of epistaxis or its method of use. Anatomical and physiological features shown and described therein FIG. 1 may be referred to as their commonly used name, their medical name, or combinations thereof. Those skilled in the art may use yet other names to describe the physiological and anatomical features shown and described therein FIG. 1.

In one aspect, bleeding may occur in the nasal cavity through the breaking, cracking, drying, or other damage to the internal epidermis of the nasal cavity at, near, or above the blood vessels as illustrated in FIG. 1. By way of example and not limitation, epidermis may dry out causing cracks due to dry air or other environmental circumstances. By way of example and not limitation, this may happen at, near, proximate or above internal maxillary artery 100, sphenopalatine artery 101, Kiesselbach's area 102, ophthalmic artery 200, posterior ethmoidal artery 201, anterior ethmoidal artery 202, other capillaries connected therein, corresponding veins, the like, and/or combinations thereof. Of particular interest to the formulation of the disclosure may be that blood vessels deep within nasal cavity C ophthalmic artery 200, posterior ethmoidal artery 201, and anterior ethmoidal artery 202 may have epidermal tissue which could otherwise be out of reach using a cotton swab method of application of a moisturizing formulation. If such epidermis continues in its dry state and persists to crack, persistent epistaxis may occur. Bleeding may occur and descend down nasal cavity C and outward to nostril O of nose N, or may descend inward toward throat T. This may cause significant pain, discomfort, irritation, inconvenience, and embarrassment to a patient persistently or chronically suffering from such a condition. Additionally, it may be difficult to stop bleeding and cause significant anxiety or even result in the need to seek medical care and attention. A nasal spray, consisting of the formulation of the disclosure, may deeply penetrate nasal cavity C in order to prevent dryness, cracking, and bleeding of ophthalmic artery 200, posterior ethmoidal artery 201, and anterior ethmoidal artery 202.

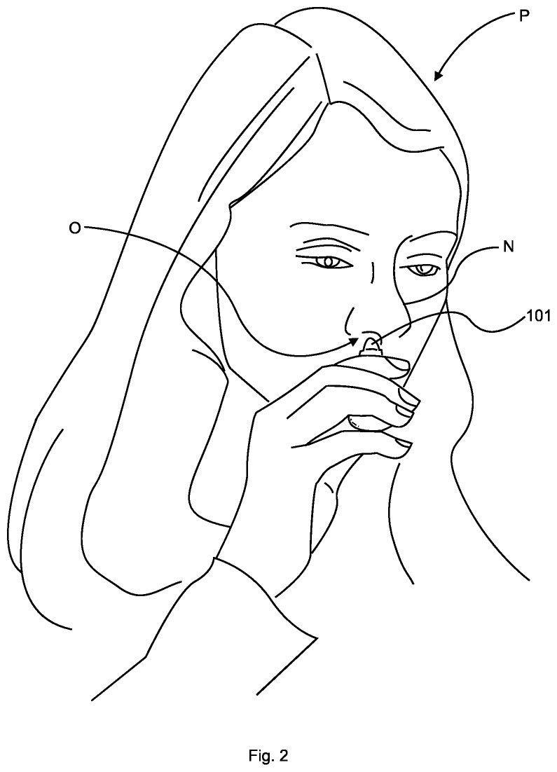

Referring now to FIG. 2 specifically, therein is illustrated a perspective view of exemplary patient P applying a nasal spray to nostril O via an exemplary nasal spray device, nasal spray bottle 10. Patient P therein is illustrated, by way of example and not limitation, bringing nasal spray bottle 10 to nose N and directing the tip of nasal spray bottle 10 toward and into nostril O. Depending on the type and configuration of nasal spray bottle 10, patient P may be instructed or may on her own volition squeeze, pump, or otherwise activate nasal spray bottle 10 to dispense, aerosolize, spray, or otherwise dispense a formulation of the disclosure into nose N via nostril O. Further depending on configuration and type of nasal spray bottle 10, patient P may lean her head forward, backward, or upright and may be in the seated, standing, or reclined position during dispensing of the formulation of the disclosure into nostril O. This step may be repeated upon another nostril O of patient P. In use, by way of example and not limitation, patient P may then may be instructed to inhale, deeply or otherwise, via either nose N or individual nostril O, in order to force the formulation of the disclosure more deeply into nasal cavity C. Patient P may be instructed to refrain from blowing of nose N for a period of time after applying of the formulation of the disclosure into nasal cavity C. Additionally, in a potentially preferred embodiment of the method of the disclosure, patient P may blow nose N in advance of application of the formulation of the disclosure.

One or more primary ingredients of a potentially preferred composition of the disclosure are present according to the following percentages by volume of the composition:

| | Amount |
|---|---|
| Ingredients | (% by Volume of the Composition) |
| MCT Oil (C8:0, C10:0, C12:0) | approximately 80% |
| Achillea millefolium preparation | approximately 10% |
| Pimpinella saxifrage preparation | approximately 10% |

In one aspect, the predominant ingredient, by volume and weight, of the disclosed formulation for epistaxis prevention may include oils derived from a fruit of a Palmaceae, namely coconut oil. The fractional weight of triglycerides in the primary ingredient of the disclosed nasal spray formulation having epistaxis prevention properties may be selected for to include those having certain properties that one skilled in the art may know to be beneficial for a nasal spray formulation. In a potentially preferred embodiment of the disclosed formulation, the formulation may include at 80% by volume a refined coconut oil mixture, fractionated and/or refined to include substantially only caprylic acid (C8:0), capric acid (C10:0), and lauric acid (C12:0) triglycerides. This formulation may be one of any number of combinations of fractionated and/or refined coconut or palm oils known, produced, manufactured, marketed, sold, and purchased as medium-chain triglyceride oil or just “MCT oil.” MCT oil may be purchased for any number of uses, including use as a dietary supplement. It is particularly useful and of note that MCT oil o this formulation generally remains in liquid form at room temperature, something that may increase its utility as a delivery mechanism in a nasal moisturizer. Additionally, it is exceedingly rare for an individual to experience irritation, allergic reaction, adverse skin condition, the like and/or combinations thereof from the application of coconut and/or MCT oil to the individual's epidermis.

In order to create a preparation of Achillea millefolium, or the Achillea millefolium preparation, first a mother tincture may be made from the whole plant of Achillea millefolium according to The Homeopathic Pharmacopoeia of the United States (HPUS) under the monograph HPUS 6102, its latest revision being Nov. 2, 2013. The monograph for Achillea millefolium HPUS 6102, including the instructions for preparation of a 1× alcohol-based tincture thereof are hereby incorporated by reference. It has long understood properties for treatment of wounds, owing its Latin name to Achilles, who was said to have used yarrow for the wounds of his friends on the battlefield. In addition to treatment of wounds, references may be found in various versions of the Materia Medica for the prevention of nosebleeds, or epistaxis. After a tincture has been made of Achillea millefolium, it may be then diluted and made alcohol free according to the instructions herein described to fully prepare a Achillea millefolium preparation.

In order to create a preparation of Pimpinella saxifrage, or the Pimpinella saxifrage preparation, first a mother tincture may be made from the root of Pimpinella saxifrage according to HPUS under the monograph HPUS 7065. The monograph for Pimpinella saxifrage HPUS 7065, including the instructions contained therein for the preparation of a 1× alcohol-based tincture thereof are hereby incorporated by reference. It is an alcohol-based tincture and references may also be found in various versions of the Materia Medica for the prevention of epistaxis and reduction in nasal dryness. After a tincture has been made of Pimpinella saxifrage, it may be then diluted and made alcohol-free according to the instructions herein described to fully prepare a Pimpinella saxifrage preparation.

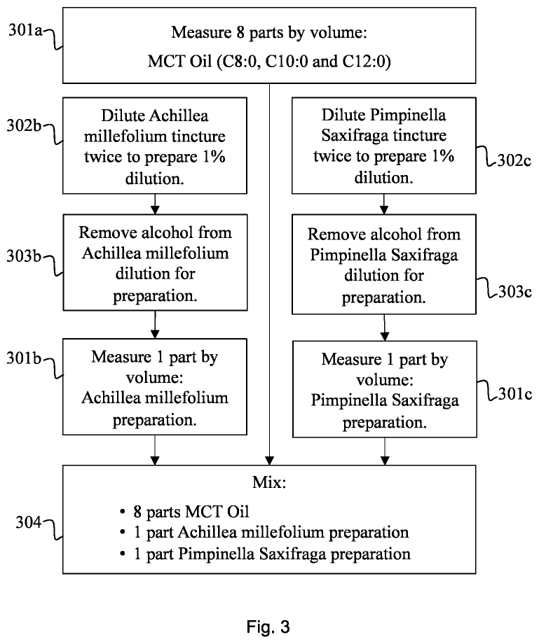

It is important to note, that in potentially preferred preparations of both an Achillea millefolium preparation and a Pimpinella saxifrage preparation, dilutions may be made of 1× preparations prepared according to the monographs HPUS 6102 and HPUS 7065, respectively. This may occur at step 302b and/or step 302c of FIG. 3. Additionally, in potentially preferred preparations of both an Achillea millefolium preparation and a Pimpinella saxifrage preparation, it may be important to remove alcohol from the herbal tinctures so as to prevent drying which may occur to the epidermis as is known by those skilled in the art to occur after the application and drying of alcohol-based preparations. This may occur at step 303b and/or step 303c. In order to dilute a 1× preparation or 1× tincture at step 302b and/or step 302c, it is important to understand the meaning of 1×, 2×, 3×, etc. dilutions in the art of herbal tincture preparations. A 1× tincture is usually the fully-concentrated tincture as specified by the homeopathic or pharmacological literature. This baseline 1× tincture is essentially undiluted. A 2× tincture or 2× dilution is 1-part (by weight or volume) the 1× tincture and 9 parts (by weight or volume) dilutant. Dilutant may include but is not limited to ethyl alcohol, other alcohols, pure or mineral water, oils, milks, the like and/or combinations thereof. Dilutants may be selected for their reaction (or lack thereof) with the 1× tincture solute(s). A 3× dilution may then be prepared from the mixing of 1-part 2× dilution with 9 parts dilutant. The second dilutant, while usually the same as the first, is not required to be the same dilutant. Dilutions can be further prepared similarly, that is for every nX dilution there are (n−1) dilution steps performed where 9 parts of dilutant are mixed with 1 part of the (n−1)× dilution to form n× dilution. Accordingly, it can be understood that an order of magnitude is reduced for each increase in “n” for each× dilution. For example, a 2× dilution would be a 10^{th} concentrated preparation or 10% concentration of 1×, a 3× dilution would be a 100^{th} concentrated preparation or 1% concentration of 1×, and so on. In a potentially preferred embodiment of an Achillea millefolium preparation, dilutions may be made accordingly to prepare a 3× dilution of a HPUS 6102 tincture. In a potentially preferred embodiment of a Pimpinella saxifrage preparation, dilutions may also be made accordingly to prepare a 3× dilution of an HPUS 7065 tincture. It may be otherwise understood that these preparations may be at 1% concentration of their corresponding 1× tinctures. In a potentially preferred embodiment of both the Achillea millefolium preparation and the Pimpinella saxifrage preparation, dilutions may be made with MCT Oil as a dilutant. This dilutant may be selected for the formulation of the disclosure due to its inclusion as an ingredient in the final formulation. Finally, in a potentially preferred embodiment, at step 303b and/or step 303c, ethyl alcohol as understood to be contained in each 1× tincture may be removed from each solution by any methods of alcohol removal known by those skilled in the art, including but not limited to boiling and/or evaporative alcohol removal.

Having described further background regarding important steps and features of the primary ingredients of a potentially preferred embodiment of the formulation of the disclosure, turning now specifically to FIG. 3, therein illustrated is a flow chart showing a potentially preferred embodiment of the basic method of preparing the formulation of the disclosure. At step 301a, a measurement of MCT Oil may be taken according to the preparation of an 8-part MCT Oil formulation. By way of example and not limitation, if 10 mL of formulation are intended to be created, one skilled in the art of making such a formulation would know to measure 8 mL of MCT Oil. Next, at step 302b and step 302c, an amount of each of Achillea millefolium tincture and Pimpinella saxifraga tincture may be measured and diluted to 1% or 3X dilution according to the principles described above. At step 303b and step 303c, alcohol may be removed from each tincture through the process(es) described above. Next, at step 301b and step 301c, a measurement may be taken to obtain the one part needed of each tincture according to the 8:1:1 ratio of the formulation. As per the example recited above, as it related to an 8 mL measurement of MCT Oil, 1 mL would be measured of each diluted and alcohol-free tincture. Finally, at step 304, a mixture or formulation may be made by mixing the ingredients obtained at step 301a, step 301b, and step 301c.

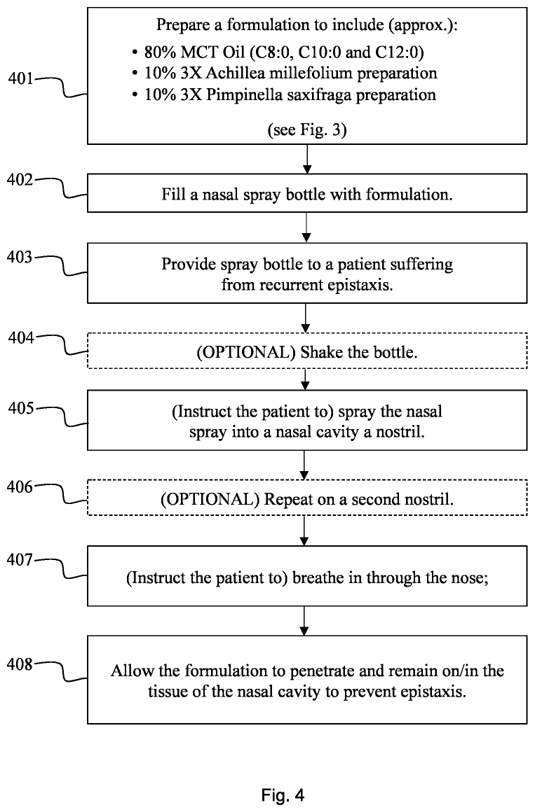

Having described how to prepare a potentially preferred embodiment of the formulation of the disclosure, turning now specifically to FIG. 4, therein illustrated is a flow chart showing a potentially preferred embodiment of the basic method of treatment for epistaxis using the formulation of the disclosure. First, at step 401, a formulation of the disclosure according to steps 301-304 of FIG. 3 may be prepared. Essentially, an 8:1:1 mixture of MCT Oil, 3× alcohol free Achillea millefolium and 3× Pimpinella saxifrage may be prepared. Next, at step 402 nasal spray bottle 10 may be filled with the formulation of step 401. Patient P may then be provided with nasal spray bottle 10 at step 403. Optionally, patient P may shake the bottle to ensure even mixture at step 404. After mixing, the spray bottle may be inserted into nostril O of patient P, or patient P may be instructed to so do, at step 405. This step may optionally be repeated, at step 406, upon a second nostril. Patient P may then in inhale deeply through the nose to ensure sufficient penetration of the formulation into the nasal cavity, or patient P may be instructed to so do, at step 407. Finally, it may be preferable for patient P to wait, or be instructed to wait, for a period of time at step 408.

It is contemplated herein that variations of the formulation of the disclosure may be made in order to accomplish the intended effect of the prevention of epistaxis. By way of example and not limitation, other formulations of MCT oil, coconut oil, palm oil, vegetable oils, the like and/or combinations thereof may be selected for these properties and substituted therein the formulation of the disclosure and may achieve the same, similar, or even preferable results. Specifically, oils may be chosen for their scent, viscosity, state at room temperature, moisture sealing properties, food safety, hypoallergenic properties, the like and/or combinations thereof. By way of example and not limitation, some or all of the approximately 80% by volume ingredient may include mineral oil, olive oil, linseed oil, tung oil, walnut oil, vegetable oil, canola oil, palm oil, walnut oil, the like and/or combinations thereof. Variations of the lower quantity ingredients may also be selected for the properties they may or may not possess. By way of example, lower quantity ingredients may include alternative mother tinctures (either diluted, undiluted, containing alcohol or alcohol free), scents and/or fragrances, essential oils, the like and/or combinations thereof.

Alternative mother tinctures, which may be substituted for a lower-quantity ingredient of the potentially preferred embodiment of the formulation, may include by way of example and not limitation, Absinthium (Grande Wormwood), Aesculus hipp. (Horse Chestnut), Agnus castus (Chaste tree), Alfalfa (California Clover), Allium cepa (Red Onion), Allium sativa (Garlic), Angelica arch. (European Angelica), Arnica (Leopardsbane), Arnica/Rhus-t/Ruta, Avena sativa (Common oat), Baptisia tinctoria (Wild Indigo), Berberis aquifolium (Mahonia aquifolium), Berberis vulgaris (Barberry), Bryonia (White Bryony), Cactus grandiflorus (Night-Blooming Cereus), Calendula (Pot Marigold), Camphor (Camphora) (Camphor purum, Camphor Officinarum), Cardiospermum (Heart seed), Carduus marianus (St Mary's Thistle, Milk Thistle), Ceanothus (New Jersey Tea), Chamomilla (German Chamomile), Chelidonium (Greater Celandine), Chimaphila umbellata (Pipsissewa), China officinalis (Peruvian Bark), Condurango (Condor Plant), Crat/Cactus/China, Curcuma longa (Turmeric, curcuma rotunda), Echinacea angustifola (Black Sampson), Echinacea purpurea (Purple coneflower), Equisetum hyemale (Scouring Rush), Euphrasia officinalis (Eyebright), Fraxinus americana (White Ash), Fucus vesiculosus (Sea Kelp), Ginkgo biloba, Ginseng (Panax ginseng), Glycyrrhiza glabra (Liquorice), Hamamelis virginica (Witch Hazel), Humulus lupulus (Hops), Hydrastis canadensis (Golden Seal), Hydrocotyle (Indian Pennywort), Hypericum (St John's wort), Hypericum/Calendula, Jonesia Ashoka (Ashoka), Ledum pal. (Marsh Tea), Lobelia inflata (Indian tobacco), Lycopodium (Club Moss), Lycopus virginicus (Bugle weed), Myrrha (Resin from Commiphora molmol), Paeonia officinalis (Peony), Passiflora (Passion Flower), Passiflora/Avena Sat/Valerian, Phytolacca Dec. (Poke root), Propolis, Prunus spinosa (Blackthorn, Sloe), Pyrethrum parthenium (Feverfew), Rhus-t/Ruta, Rosmarinus (Rosemary), Ruta graveolens (Rue), Sabal serrulata (Saw Palmetto), Salvia officinalis (Sage), Sequoia (Seq dendron Gig) (Sequoiadendron Giganteum), Spartium scoparium (English Broom), Spiritus Quercus Gland. (Acorns of Quercus rober, Quercus Glandium Spiritus), Stellaria (Chickweed), Symphytum (Comfrey), Syzygium jambolanum (Jambol seeds), Taraxacum (Dandelion), Thuja occidentalis (Arbor vitae), Thymus serpyllum (Thymus serpyllumwild Thyme), Urtica urens (Dwarf nettle), Urtical (Urtica Urens/Calendula), Valerian officinalis (Valeriana), Verbena officinalis (Common Vervain), Zingiber (Ginger), the like and/or combinations thereof.

Essential oils, which may be substituted for a lower-quantity ingredient of the potentially preferred embodiment of the formulation, may include, but are not limited to, Agar oil or oodh, Ajwain oil, Angelica root oil, Anise oil, Asafoetida oil, Balsam of Peru, Basil oil, Bay oil, Bergamot oil, Black pepper oil, Buchu oil, Birch oil, Camphor oil, Cannabis flower oil, Calamodin oil or calamansi essential oil, Caraway seed oil, Cardamom seed oil, Carrot seed oil, Cedar oil (or cedarwood oil), Chamomile oil, Calamus oil, Cinnamon oil, Cistus ladanifer leaves and flowers, Citron oil, Citronella oil, Clary Sage oil, Coconut oil, Clove oil, Coffee oil, Coriander oil, Costmary oil (bible leaf oil), Costus root oil, Cranberry seed oil, Cubeb oil, Cumin seed oil/black seed oil, Cypress oil, Cypriol oil, Curry leaf oil, Davana oil, Dill oil, Elecampane oil, Elemi oil, Eucalyptus oil, Fennel seed oil, Fenugreek oil, Fir oil, Frankincense oil, Galangal oil, Galbanum oil, Garlic oil, Geranium oil, Ginger oil, Goldenrod oil, Grapefruit oil, Henna oil, Helichrysum oil, Hickory nut oil, Horseradish oil, Hyssop, Idaho-grown Tansy, Jasmine oil, Juniper berry oil, Laurus nobilis, Lavender oil, Ledum, Lemon oil, Lemongrass, Lime, Litsea cubeba oil, Linalool, Mandarin, Marjoram, Melissa oil (Lemon balm), Mentha arvensis oil, Moringa oil, Mountain Savory, Mugwort oil, Mustard oil, Myrrh oil, Myrtle, Neem oil or neem tree oil, Neroli, Nutmeg oil, Orange oil, Oregano oil, Orris oil, Palo Santo, Parsley oil, Patchouli oil, Perilla essential oil, Pennyroyal oil, Peppermint oil, Petitgrain, Pine oil, Ravensara, Red Cedar, Roman Chamomile, Rose oil, Rosehip oil, Rosemary oil, Rosewood oil, Sage oil, Star anise oil, Sandalwood oil, Sassafras oil, Savory oil, Schisandra oil, Spearmint oil, Spikenard, Spruce oil, Star anise oil, Tangerine, Tarragon oil, Tea tree oil, Thyme oil, Tsuga oil, Turmeric, Valerian, Warionia, Vetiver oil (khus oil), Western red cedar, Wintergreen, Yarrow oil, Ylang-ylang, the like, and/or combinations thereof.

It is contemplated herein that in addition to the treatment and/or prevention of epistaxis, a formulation of the disclosure may offer the included and/or additional benefits moisturizing the epidermis of the nasal cavity and ceasing, clotting, or otherwise stopping the bleeding from the nose during an epistaxis event. Each of these benefits may be important to patients who do not suffer recurrent and/or frequent epistaxis events. It is further contemplated herein that a formulation and/or preparation of the disclosure may be applied through a number of mechanisms, including nasal spray bottle(s) 10, but also including by way of example and not limitation, droppers, cotton swabs (e.g. Q-TIPS® cotton swabs), Tx360EU nasal applicator, syringe, nasal irrigator, metered spray pump, non-metered spray pump, mist-type nasal sprayers (e.g. FLONASE® SENSIMIST® nasal spray bottle), the like and/or combinations thereof. Furthermore, should the epistaxis prevention properties of the formulation be found to extend further to the prevention of bleeding, cracking, drying, etc. of other areas of epidermis, yet other application methods may be favorable. Finally, it is contemplated herein that the formulation of the disclosure may further treat epistaxis, or other conditions, found in other, non-human, animals.

The foregoing description and drawings comprise illustrative embodiments. Having thus described exemplary embodiments, it should be noted by those skilled in the art that the within disclosures are exemplary only, and that various other alternatives, adaptations, and modifications may be made within the scope of the present disclosure. Merely listing or numbering the steps of a method in a certain order does not constitute any limitation on the order of the steps of that method. Many modifications and other embodiments will come to mind to one skilled in the art to which this disclosure pertains having the benefit of the teachings presented in the foregoing descriptions and the associated drawings. Although specific terms may be employed herein, they are used in a generic and descriptive sense only and not for purposes of limitation. Accordingly, the present disclosure is not limited to the specific embodiments illustrated herein, but is limited only by the following claims.

What is claimed is:

1. A nasal moisturizing formulation, the formulation comprising a mixture of approximately 80% by volume a medium chain triglyceride coconut oil, said medium chain triglyceride coconut oil is fractionated to include substantially only caprylic acid, capric acid, and lauric acid, approximately 10% by volume an alcohol-free and two-times diluted tincture of Achillea millefolium, and approximately 10% by volume an alcohol-free and two-times diluted tincture of Pimpinella saxifraga.

2. The formulation of claim 1, wherein said alcohol-free and two-times diluted tincture of Achillea millefolium is diluted from a mother tincture of HPUS 6102.

3. The formulation of claim 1, wherein said alcohol-free and two-times diluted tincture of Pimpinella saxifraga is diluted from a mother tincture of HPUS 7065.

4. The formulation of claim 2, wherein said alcohol-free and two-times diluted tincture of Pimpinella saxifraga is diluted from a mother tincture of HPUS 7065.

5. The formulation of claim 4, wherein said alcohol-free and two-times diluted tincture of Achillea millefolium and said alcohol-free and two-times diluted tincture of Pimpinella saxifraga are first diluted and then boiled to remove the alcohol prior to being added into the formulation.

6. The formulation of claim 1, wherein the formulation is contained within a nasal spray bottle.

7. The formulation of claim 1, wherein a dilutant used to dilute said alcohol-free and two-times diluted tincture of Achillea millefolium and said alcohol-free and two-times diluted tincture of Pimpinella saxifrage is said medium chain triglyceride coconut oil.

8. The formulation of claim 1, wherein the nasal moisturizing formulation prevents a nasal dryness-related epistaxis.

9. A nasal spray bottle filled with the formulation of claim 1.

10. A method of preparing a formulation for nasal moisturization, the method comprising:
measuring eight parts by volume a medium chain triglyceride coconut oil to obtain a measurement of MCT oil;
diluting a tincture of Achillea millefolium twice using one part said tincture of Achillea millefolium and nine parts of a dilutant to prepare a Achillea millefolium dilution;
diluting a tincture of Pimpinella saxifraga twice using one part said tincture of Pimpinella saxifraga and nine parts of said dilutant to prepare a Pimpinella saxifraga dilution;
removing alcohol from said Achillea millefolium dilution and said Pimpinella saxifraga dilution to prepare a Achillea millefolium preparation and a Pimpinella saxifraga preparation;
measuring one part by volume of each of said Achillea millefolium preparation and said Pimpinella saxifraga preparation to obtain a measurement of Achillea millefolium preparation and a measurement of Pimpinella saxifraga preparation; and
mixing said measurement of MCT oil with said measurement of Achillea millefolium preparation and said measurement of Pimpinella saxifraga preparation to form the formulation.

11. The method of claim 10, wherein said medium chain triglyceride coconut oil is fractionated to include substantially only caprylic acid, capric acid, and lauric acid.

12. The method of claim 10, wherein said tincture of Achillea millefolium is a mother tincture of HPUS 6102 and wherein said tincture of Pimpinella saxifraga is a mother tincture of HPUS 7065.

13. The method of claim 10, wherein alcohol is removed from said Achillea millefolium dilution and said Pimpinella saxifraga dilution by a method of alcohol removal from a group of methods, the methods comprising boiling and evaporation.

14. The method of claim 10, wherein each diluting step is performed by adding said medium chain triglyceride coconut oil.

15. A method of nasal moisturization of a patient, the method comprising:
providing a formulation comprising a mixture of approximately 80% by volume a medium chain triglyceride coconut oil, approximately 10% by volume an alcohol-free and two-times diluted tincture of Achillea millefolium, and approximately 10% by volume an alcohol-free and two-times diluted tincture of Pimpinella saxifrage;
filling a nasal spray bottle with said formulation; and
causing said nasal spray bottle to spray said formulation into a first nostril of the patient.

16. The method of claim 15, wherein said medium chain triglyceride coconut oil is fractionated to include substantially only caprylic acid, capric acid, and lauric acid.

17. The method of claim 15, wherein said alcohol-free and two-times diluted tincture of Achillea millefolium is diluted from a mother tincture of HPUS 6102.

18. The method of claim 15, wherein said alcohol-free and two-times diluted tincture of Pimpinella saxifraga is diluted from a mother tincture of HPUS 7065.

19. The method of claim 17, wherein said alcohol-free and two-times diluted tincture of Pimpinella saxifraga is diluted from a mother tincture of HPUS 7065.

20. The method of claim 15, further comprising the step of causing said nasal spray bottle to spray said formulation into a second nostril of the patient.
